(19) United States
(12) Patent Application Publication
(10) Pub. No.: US 2015/0223749 A1
(43) Pub. Date: Aug. 13, 2015

(54) SKIN CONDITION EVALUATION APPARATUS AND SKIN CONDITION EVALUATION METHOD USING THE SAME

(71) Applicant: Seoul Viosys Co., Ltd., Ansan-si (KR)

(72) Inventors: Stella Park, Seoul (KR); Daewoong Suh, Seongnam-si (KR)

(21) Appl. No.: 14/620,125

(22) Filed: Feb. 11, 2015

(30) Foreign Application Priority Data
Feb. 11, 2014 (KR) 1020140015194

(51) Int. Cl. A61B 5/00

(52) U.S. Cl. CPC A61B 5/443; A61B 5/0071; A61B 5/0077; A61B 5/4875

(57) ABSTRACT

Provided is a skin condition evaluation method. The skin condition evaluation method may include preparing a light source including one or more LEDs, irradiating light onto skin using the light source, and receiving light emitted through the skin using a light detector.

### PRIORITY CLAIM AND CROSS-REFERENCE TO RELATED APPLICATIONS

This patent document claims priority from and benefits of Korean Patent Application No. 10-2014-0015194, entitled “APPARATUS FOR EVALUATING SKIN CONDITION AND METHOD OF EVALUATING SKIN CONDITION USING THE SAME” and filed on Feb. 11, 2014, which is incorporated herein by reference in its entirety.

### TECHNICAL FIELD

This patent document relates to a skin condition evaluation apparatus and a skin condition evaluation method using the same.

### BACKGROUND

As we become more interested in personalized healthcare, techniques for evaluating and managing health conditions through continuous monitoring become increasingly important. Skin, as our largest organ, presents various metabolites and changes through both internal and external stimuli. As one example, when a person has a bad complexion, the person may have a blood-flow disorder or digestive disorder. As another example, when a person has skin troubles, the person may experience stress.

### SUMMARY

Various implementations of the disclosed technology provide an apparatus and method for easily evaluating a skin condition using a non-destructive method. Further, the implementations of the disclosed technology provide an skin evaluation apparatus and a skin evaluation method capable of analyzing specific component concentration existing in epidermis of a skin using a light analysis method, and removing the influence of the analyzed specific component from light information. Thus, it enables to determine reliable internal skin information without being affected by a specific component.

In one aspect, a skin condition evaluation method is provided to include providing a light source including one or more LEDs, irradiating light from the light source onto a skin, responsive to the irradiating, receiving, by a light detector, light emitted through the skin including ultraviolet (UV) light; calculating, based at least partly on the received light emitting through the skin, an amount of a component distributed in the skin.

In some implementations, based on a presence of the component in an epidermis of the skin, the calculating of the amount of the specific component can include: calculating a diffuse reflectance R of a semi-infinite layer based at least partly on information on light received by the light detector; calculating a single scattering albedo ω(λ) from the diffuse reflectance R of the semi-infinite layer using an equation,

$$R = ( 1 - {ρ}_{01} ) [ 1 - {\hat{ρ}}_{10} ( ω ) ] \frac{{\hat{R}}_{d} ( ω )}{1 - {\hat{ρ}}_{10} ( ω ) {\hat{R}}_{d} ( ω )}$$

where ρ₀₁ is the specular reflection of incident radiation by the surrounding/medium interface, \hat{ρ}₁₀ is semi-empirical hemispherical-hemispherical reflectivity, and \hat{R}_{d} is the semi-empirical diffuse reflectance of semi-infinite layer when exposed to diffuse irradiation; calculating an absorption coefficient μₑₚᵢ of the epidermis from the single scattering albedo ω(λ) using an equation,

$$ω ( λ ) = \frac{{μ}_{s , tr} ( λ )}{{μ}_{s , tr} ( λ ) + {μ}_{epi}} ,$$

where μ_{s, tr} is a transport scattering coefficient; and calculating a volume fraction fₛₚₑ of specific component in the epidermis from the absorption coefficient μₑₚᵢ of the epidermis using an equation,

μₑₚᵢ=μₛₚₑ(λ)fₛₚₑ+μ_{back}(λ)(1−fₛₚₑ), where μback is background absorption of human flesh.

In some implementations, the UV light has a peak wavelength of 300 nm to 400 nm. In some implementations, the irradiating of the light includes emitting by the one or more LEDs one or more lights including UV light, visible light, or infrared light, and the skin condition evaluation method further comprises removing a contribution of the amount of the component to the light received by the light detector. In some implementations, the irradiating of the light onto the skin comprises providing different wavelengths of light emitted from a plurality of LEDs, and the skin condition evaluation method further comprises removing a contribution of the amount of the specific component to the light received by the light detector. In some implementations, the receiving of the light emitted through the skin comprises receiving one or more of a reflected light spectrum, a fluorescence spectrum, or a scattering light spectrum from the skin. In some implementations, the skin condition evaluation method further comprises calculating a spectrum absorbed by the component of the skin based on the received light, using an arithmetic unit. In some implementations, the irradiating of the light onto the skin comprises emitting by the one or more LEDs a polarizing light. In some implementations, the receiving of the light emitted through the skin comprises receiving polarized light of the light emitted from the skin. In some implementations, the skin condition evaluation method further comprises: capturing an image of the skin, the captured image of the skin being divided into regions; mapping information associated with the received light from the light detector to the respective regions; and displaying the information mapped to the respective regions of the skin image, using a display device. In some implementations, the component is melanin.

In another aspect, a skin condition evaluation apparatus is provided to include: a light source comprising one or more LEDs to irradiate light including UV light onto a skin; a light detector configured to receive light emitted through the skin responsive to the irradiated light; and an arithmetic unit configured to calculate a spectrum absorbed by one or more components of the skin based at least partly on the received light, wherein each of the one or more LEDs is independently driven to irradiate the light and the arithmetic unit is configured to calculate an amount of the one or more components of the skin based on the light received by the light detector, and remove a contribution of the calculated amount of the one or more components to the received light.

In some implementations, the light detector receives one or more of a reflected light spectrum, a fluorescence spectrum, or a scattering light spectrum from the skin. In some implementations, the light source comprises a polarizing device to polarize the light emitted from the one or more LEDs such that the polarized light is irradiated on the skin. In some implementations, the light detector comprises a polarizing device to receive the polarized light of the light emitted from the skin. In some implementations, an angle between a polarization direction of the polarizing device of the light source and a polarization direction of the polarizing device of the light detector is adjustable. In some implementations, the skin condition evaluation apparatus further comprises a camera configured to capture an image of the skin, wherein the arithmetic unit maps the light information received by the light detector to a plurality of regions of the skin image captured through the camera, and the skin condition evaluation apparatus further comprises a display device configured to display the light information mapped to the respective regions of the skin image. In some implementations, the light detector and the camera are integrated with each other. In some implementations, the one or more LEDs are symmetrically or radially arranged with respect to the light detector. In some implementations, the one or more components include melanin.

Those and other aspects of the disclosed technology and their implementations and variations are described in greater detail in the drawings, the description and the claims.

### BRIEF DESCRIPTION OF THE DRAWINGS

FIG. 1 is a flowchart schematically illustrating a skin condition evaluation method according to some implementations of the disclosed technology.

FIGS. 2A to 2C are graphs illustrating light absorption coefficients, light absorption intensities, and fluorescence intensities of skin components.

FIG. 3 is a block diagram schematically illustrating a skin condition evaluation apparatus according to some implementations of the disclosed technology.

FIGS. 4A and 4B schematically illustrate the operation of the skin condition evaluation apparatus according to some implementations of the disclosed technology.

FIG. 5 is a schematic view of the skin condition evaluation apparatus according to some implementations of the disclosed technology.

FIGS. 6 and 7 are a perspective view and a cross sectional view of a skin condition evaluation apparatus including LEDs according to some implementations of the disclosed technology.

### DETAILED DESCRIPTION

Exemplary implementations will be described below in more detail with reference to the accompanying drawings. The disclosure may, however, be embodied in different forms and should not be constructed as limited to the embodiments set forth herein. Throughout the disclosure, like reference numerals refer to like parts throughout the various figures and embodiments.

This patent document provides various implementations for a skin condition evaluation apparatus and a skin condition evaluation method using the same.

A skin condition can be evaluated through a visual or tactile method. In one aspect, skin elasticity may be visually determined through an optical microscope, and skin water content may be tactually determined. For example, skin care information can be obtained by applying a voltage to a user's skin, detecting a current signal flowing in the user's skin, measuring the skin water content and sweat duct activity of the user, and calculating water content information of the skin.

Skin monitoring should be consistently conducted to evaluate a health condition. To accomplish consistent monitoring of a skin condition, the evaluation apparatus and method should be convenient without giving an unpleasant feeling to a person whose skin is to be evaluated.

In an example of related technologies, a multi-functional digital skin imaging apparatus can includes a light source, a CCD (Charge Coupled Device) camera, and a rotary filter wheel stage having one or more optical filters. As the rotary filter wheel stage is rotated, one of the optical filters with a wavelength selection function is selected and positioned in front of the lens of the CCD camera. However, the use of the filter can increase the overall cost of the multi-functional digital skin imaging apparatus. Also, an image from the apparatus may be distorted by the influence of secondary characteristics of the filter. Furthermore, since the skin imaging apparatus must have light passed through the filter, the skin imaging apparatus can have difficulties in measuring a wavelength with a low intensity. Furthermore, since the skin imaging apparatus is large and heavy, it is likely impossible for individuals to carry the skin imaging apparatus.

In another example of related technologies, a camera for examining a skin condition can include an ultraviolet (UV) light filter and a polarizing filter. The UV light filter passes a predetermined range of wavelengths, and the polarizing filter separates light generated in reaction to the sebum of skin. The camera may divide the state of the sebum into specific colors in order to determine the state of the sebum. However, the wavelength used in the camera for examining a skin condition is limited to a specific wavelength which responds to sebum, and the price of the camera inevitably increases because the UV light filter is used. Furthermore, the camera also has the above-described problems such as the image distortion and the limit of measurable wavelengths.

Various implementations of the disclosed technology provide a skin condition evaluation method and a skin condition evaluation apparatus to evaluate a skin condition with an improved accuracy.

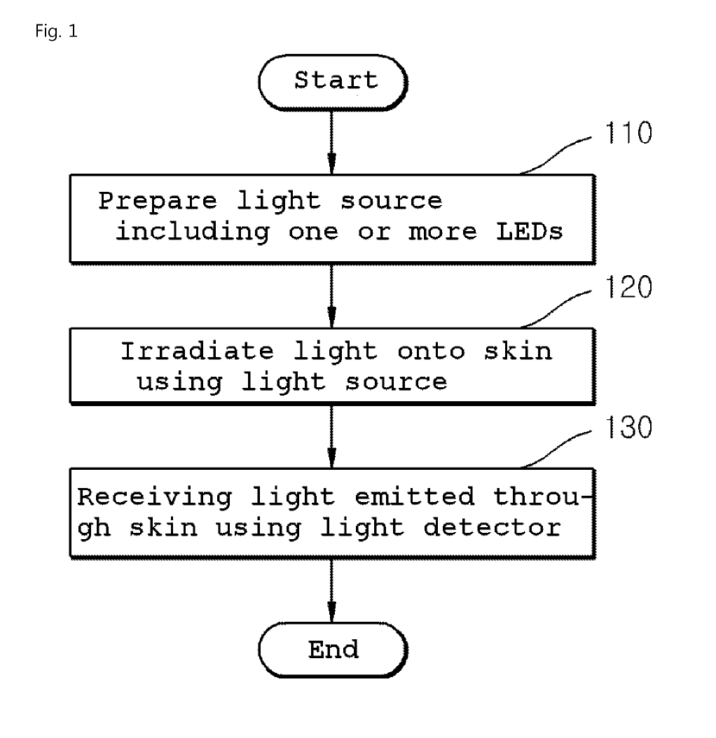

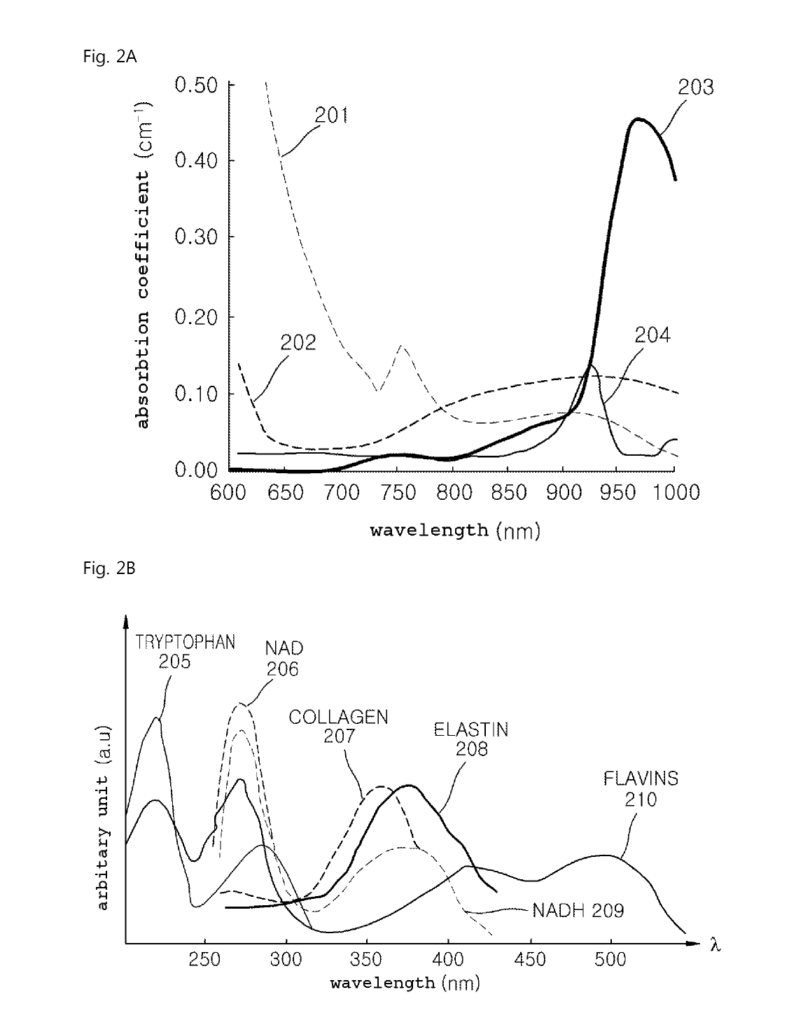

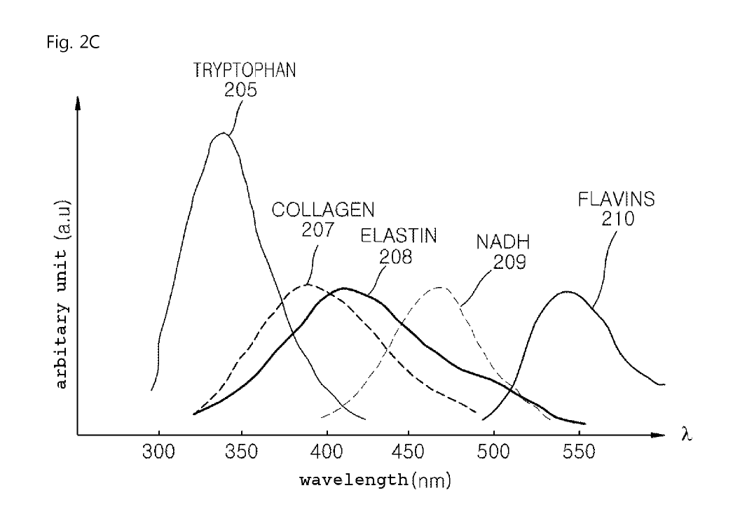

FIG. 1 is a flowchart schematically illustrating a skin condition evaluation method according to some implementations of the disclosed technology. FIGS. 2A to 2C are graphs illustrating light absorption coefficients, light absorption intensities, and fluorescence intensities of skin components.

At step 110, a light source including one or more light emitting diodes (LEDs) may be prepared. In some implementations, the one or more LEDs may irradiate or provide one or more lights having different wavelengths that correspond to UV light, visible light, or infrared light onto a skin. The one or more lights irradiated by the one or more LEDs can act as a probing light to obtain information on various components in the skin with different light absorption characteristics.

At step 120, light (e.g., a probing light) may be irradiated onto skin using the light source. In some implementations, a plurality of LEDs may emit different wavelengths of light. Since the LEDs have the property of providing a peak wavelength of light with a narrow spectrum half width, when a light detector is provided to detect light emitted through the skin, the wavelength of light received by the light detector may have a narrow spectrum half width corresponding to the peak wavelength. As a result, the light emitted from the LEDs may improve the reliability of light analysis. The skin may include a part of the human body, such as face, hand, or foot, and indicate a part of skin exposed to the outside.

At step 130, responsive to the light irradiated by the light source, the light emitted through the skin may be received by the light detector. In some implementations, the step of receiving the light emitted through the skin by the light detector may include receiving a reflected light spectrum, fluorescence spectrum, and/or scattering light spectrum emitted through the skin responsive to the probing light irradiated by the light source. At this time, each spectrum may be separately received, or two or more spectrums may be concurrently received.

Although not illustrated in the flowchart, the skin condition evaluation method may further include calculating, based on the received light, a spectrum absorbed by components of the skin using an arithmetic unit. Information associated with the received light is affected by certain components existing in epidermis of a skin. The disclosed technology takes into account the effects of the components in the epidermis of the skin in determining the internal condition of the epidermis. In this implementation, the absorption spectrum is calculated, which indicates the light absorbed by the components after being irradiated onto the skin. By calculating the absorbance of the components, which can be used to represent the skin condition as will be discussed later, it becomes possible to determine the existences and concentrations of the components within the skin.

An indicator of a skin condition may include an oxidation degree, a hydration degree, or a collagen level and the like. The oxidation degree may be based on a relative concentration of oxyhemoglobin and deoxyhemoglobin, for example. The hydration degree may be based on water content, for example. FIG. 2A illustrates how absorption coefficients of deoxyhemoglobin 201, oxyhemoglobin 202, water 203, and lipid 204 change as the wavelength varies. FIG. 2A indicates that the deoxyhemoglobin 201, the oxyhemoglobin 202, the water 203, and the lipid 204 have different absorption wavelength bands. Furthermore, FIG. 2B indicates that tryptophan 205, oxidized Nicotinamide Adenine Dinucleotide (NAD+) 206, collagen 207, elastin 208, reduced form of NAD (NADH) 209, and flavins 210 have different light absorption intensities as the wavelength of incident light varies. NAD is a type of coenzyme found in a cell. Through the above-described step 130, an absorption spectrum of light emitted from the skin may be calculated based on the light emitted through the skin received by the light detector. The received light emitted through the skin include a reflected light spectrum, fluorescence spectrum, and/or scattering light spectrum emitted from the skin can be used to determine the absorption spectrum of light emitting from the skin. Then, the wavelength of the calculated absorption spectrum may be compared to the graphs in FIGS. 2A and 2B which illustrate the absorption coefficients and intensities of the components for various wavelengths of light. By doing so, properties of the components existing in the skin, for example, a kind, a concentration, etc., can be determined. In this way, the skin condition can be evaluated.

As another example, the indicator such as the oxidation degree, the hydration degree, or the collagen level may be acquired from the wavelength and intensity of light which is emitted in the form of fluorescence after the light is absorbed, as illustrated in FIG. 2C. FIG. 2C indicates that tryptophan 205, NAD+ 206, collagen 207, elastin 208, NADH 209, and flavins 210 emit different wavelength bands of fluorescence. Through the above-described step 130, the fluorescence spectrum of light emitted from the skin may be calculated based on the light emitted through the skin received by the light detector. The received light emitted through the skin include a reflected light spectrum, fluorescence spectrum, and/or scattering light spectrum emitted from the skin can be used to determine the absorption spectrum of light emitting from the skin. Then, the wavelength of the calculated fluorescence spectrum may be compared to the graph in FIG. 2C which illustrates the fluorescence intensities of the components for various wavelengths of light. By doing so, properties of the components existing in the skin, for example, calculate the kinds, a concentration, etc. can be determined. In this way, the skin condition can be evaluated.

When light is irradiated to examine the components within the skin, all of the light must pass through the epidermis of the skin. For example, visible light and infrared light having a relatively long wavelength penetrate into the human body through the skin. Meanwhile, ultraviolet light having a relatively short wavelength would not penetrate deep into the skin but mainly be absorbed or scattered at epidermis.

There are some specific components which exist differently in the epidermis of the skin. For example, melanin exists in the epidermis with a concentration that significantly changes depending on the race, individual characteristics, or environment in which each individual is placed.

Thus, the properties of the components within the skin, for example, the kinds and concentrations of the components within the skin affect the property of light emitted through the skin. For example, the property of light emitted through the skin may be changed according to the amount of specific component in the epidermis.

For this reason, the skin condition evaluation method disclosed in this patent document includes first analyzing a specific component, for example, the concentration of the specific component in the epidermis of a body part corresponding to an inspection object, and removing the influence of the analyzed specific component from subsequent processes including light irradiation and analysis. Thus, it is possible to accurately measure the components within the skin.

According to some implementations of the disclosed technology, UV light may be firstly irradiated onto skin to be evaluated. In some implementations, the UV light may have an ultraviolet A (UVA) range or a peak wavelength range of 300 nm to 400 nm. Since most of UV light in such a wavelength range is absorbed or scattered at the epidermis or dermis, the UV light can be effectively used to determine the amount of specific component in the epidermis.

After the UV light is irradiated onto skin from the light source, the UV light emitted through the skin may be received by the light detector. The amount of specific component distributed in the epidermis may be calculated based on the received light.

The epidermis is composed mainly of dead cells, keratinocytes, melanocytes, and langerhans. Melanocytes synthesize melanin which is the skin protein that dominates light absorption in the epidermis. The absorption coefficient of the epidermis μₑₚᵢ can be expressed as

μₑₚᵢ=μₘₑₗ(λ)fₘₑₗ+μ_{back}(λ)(1−fₘₑₗ) Equation 1

where fₘₑₗ is the volume fraction of melanocytes in the epidermis and μ_{back} is the background absorption of human flesh given by μ_{back}(λ)=7.84*10⁸λ^{-3.255}.

Furthermore, the absorption coefficient of a single melanocyte as a function of wavelength has been approximated as μₘₑₗ(λ)=6.60*10¹¹λ^{-3.33}, where λ is expressed in nanometers and μ_{back} and μₘₑₗ are in cm⁻¹.

The contribution of scattering to the overall extinction in the epidermis is represented by the single-scattering albedo ω(λ) expressed as

$$ω ( λ ) = \frac{{μ}_{s , tr} ( λ )}{{μ}_{s , tr} ( λ ) + {μ}_{epi}}$$

where μ_{s, tr} is the transport scattering coefficient.

Diffuse reflectance of a semi-infinite layer is expressed as

$$R = ( 1 - {ρ}_{01} ) [ 1 - {\hat{ρ}}_{10} ( ω ) ] \frac{{\hat{R}}_{d} ( ω )}{1 - {\hat{ρ}}_{10} ( ω ) {\hat{R}}_{d} ( ω )} \qquad Equation 3$$

where ρ₀₁ is the specular reflection of incident radiation by the surrounding/medium interface, \hat{ρ}₁₀ is semi-empirical hemispherical-hemispherical reflectivity and \hat{R}_{d} is the semi-empirical diffuse reflectance of the semi-infinite layer when exposed to diffuse irradiation.

Through Equations 1 to 3, information on melanin, which is one example of specific components in epidermis, can be obtained. At this time, these equations can be applied only in the UV light region or near-UV light region.

The diffuse reflectance R of the semi-infinite layer may be acquired from the light received by the light detector, the single scattering albedo ω(λ) may be calculated through Equation 3, the absorption coefficient μₑₚᵢ of the epidermis may be calculated from the single scattering albedo ω(λ) through Equation 2, and the volume fraction of melanin in the epidermis fₘₑₗ may be calculated from the absorption coefficient μₑₚᵢ of the epidermis through Equation 1. Then, the melanin, which is a kind of specific component in the epidermis, can be analyzed.

As discussed above, the arithmetic unit is configured to calculate the amount of specific component. Further, the arithmetic unit is configured to remove the influence on the received light by the specific component.

In some implementations, the method may further include preparing a camera, and capturing an image of the skin using the camera. The camera may include an optical diode and an image sensor, for example. The camera may capture an image of light which is emitted from the skin after light is irradiated onto the skin from the light source, while excluding light extinction such as light absorbed or scattered by the skin. Such an image may be regularly and repetitively captured and stored in a database to accumulate information on the light extinction by the skin. As described above, the absorbed light of the extinction of light by the skin is related to an oxidation degree, a hydration degree, or a collagen level in the skin. Thus, the changes of the captured images may be periodically monitored to determine the change of the components within the skin.

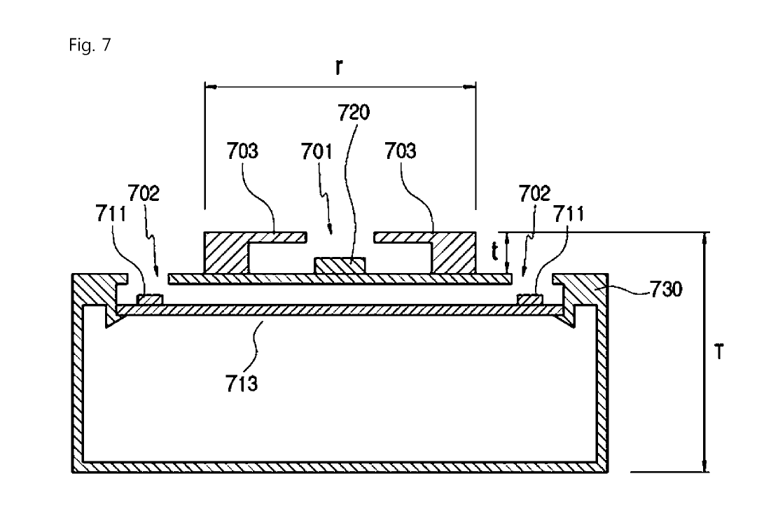

A captured image of skin to be evaluated may be divided into a plurality of detection regions. Then, the light detector may be used to map the received light information to each of the detection regions of the skin. Then, the light information mapped to each of the detection regions of the skin may be displayed on a display device. The light detector and the camera may be implemented as one device, like a light detector 720 of FIGS. 6 and 7.

According to other implementations, the light source may include a polarizing device. The light source may polarize the light emitted from the LED using the polarizing device. The light detector may also include a polarizing device, and selectively receive polarized light of the light emitted from the skin using the polarizing device. For this operation, the polarizing device may include a polarizing filter arranged at a light emitting unit of the light source and a light receiving unit of the light detector. As such, the light source may irradiate the polarized light onto the skin, and the light detector may selectively receive only the polarized light, thereby excluding noise caused by non-polarized light in the natural environment.

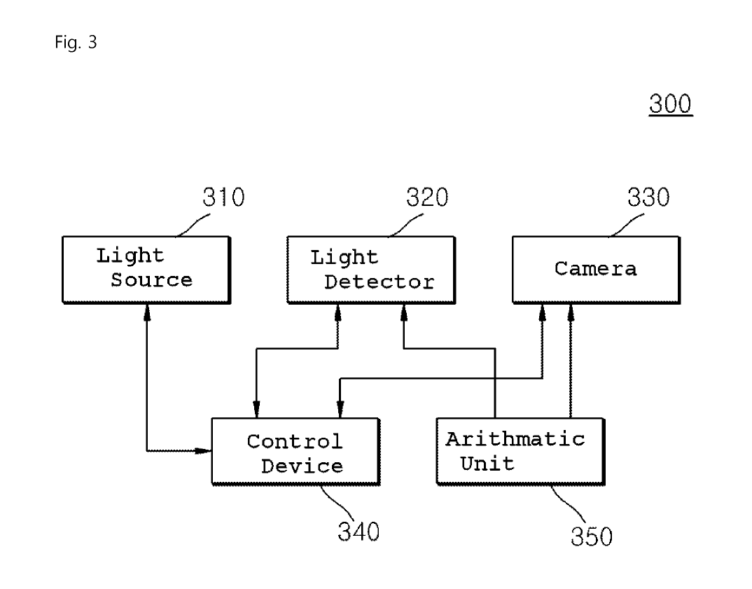

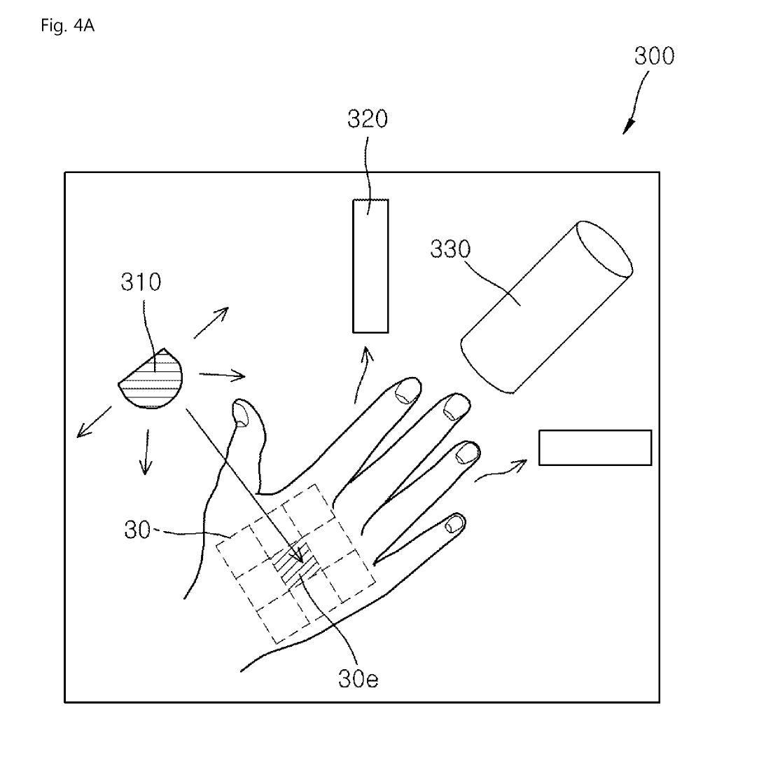

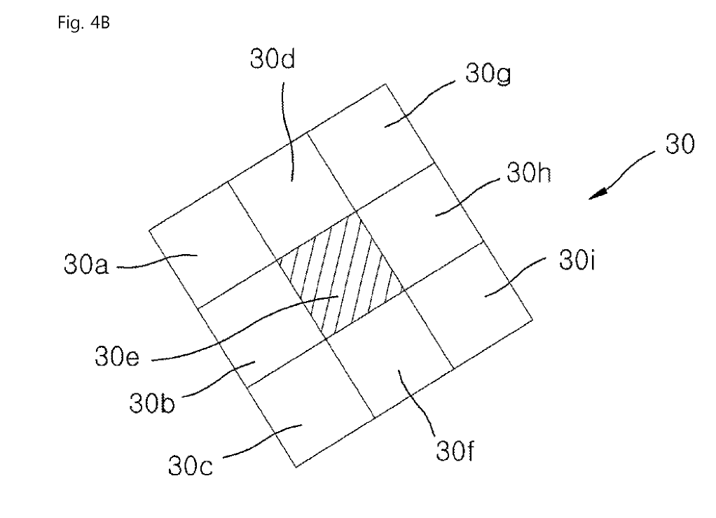

FIG. 3 is a block diagram schematically illustrating a skin condition evaluation apparatus according to some implementations of the disclosed technology. FIGS. 4A and 4B schematically illustrate the operation of the skin condition evaluation apparatus according to some implementations of the disclosed technology. Specifically, FIG. 4B is an expanded view of a plurality of detection regions obtained by dividing skin to be evaluated in FIG. 4A.

Referring to FIG. 3, the skin condition evaluation apparatus 300 may include a light source 310 and a light detector 320. Furthermore, the skin condition evaluation apparatus 300 may include a camera 330, a control device 340, and an arithmetic unit 350.

Referring to FIGS. 3, 4A, and 4B, the light source 310 may include one or more LEDs. For example, the LED may provide one or more lights selected from or including UV light, visible light, or infrared light. The light source 310 may emit different wavelengths of light using the LEDs. The LED can provide a peak wavelength of light with a narrow spectrum half width, and the wavelength of light received by the light detector 320 may have a narrow spectrum half width corresponding to the peak wavelength. Thus, when the light provided by the LED is used, reliability of analysis can be improved.

The light source 310 may irradiate the above-described light onto the skin to be evaluated. In some implementations, the skin may include a plurality of detection regions 30. FIG. 4B shows first to ninth detection regions 30a to 30i. The light source 310 may irradiate light while sequentially or non-sequentially scanning the first to ninth detection regions 30a to 30i. The light detector 320 or the camera 330 may receive lights emitted from the respective detection regions 30a to 30i. In some implementations, the light detector 320 or the camera 330 receives lights emitted from the detection regions while light is irradiated from the light source 310.

In another implementation, the light source 310 may collectively irradiate onto all of the detection regions 30, and the light detector 320 or the camera 330 may receive lights emitted from the respective detection regions 30.

The light detector 320 may include an optical diode, and receive light emitted through the detection regions 30 of the skin. The light detector 320 may receive one or more of a reflected spectrum, a fluorescence spectrum, or a scattering light spectrum that are emitted from the detection regions 30, in response to the light irradiated onto the detection regions 30 by the light source 310. Then, the arithmetic unit 350 illustrated in FIG. 3 may calculate a spectrum absorbed by the components of the skin, using the above-described spectrum information.

The camera 330 may capture an image of the skin. In some implementations, the camera 330 may include an optical diode and an image sensor, and obtain images of the above-described detection regions 30 of the skin.

In some implementations, each of the light source 310, the light detector 320, and the camera 330 may include a polarizing device (not illustrated). For example, the light source 310 may change light emitted from the LED into polarized light using the polarizing device, and irradiate the polarized light onto the skin. Similarly, the light detector 320 and the camera 330 may include a polarizing device, and selectively receive polarized light of the light emitted from the skin. Thus, the light from the natural environment and the light irradiated by the light source 310 may be distinguished from each other. The skin condition evaluation apparatus can be configured such that only the light generated by the light source 310 can be received to guarantee the reliability of the analysis result.

The arithmetic unit 350 may exchange calculation information and control signals with the light source 310, the light detector 320, the camera 330, and the control device 340. In some implementations, the arithmetic unit 350 may obtain information of light emitted from the light source 310 and information of light received by the light detector 320 or the camera 330, process the obtained information, and calculate an evaluation result related to the skin condition.

As one example, the arithmetic unit 350 may store various information on light in a database. The arithmetic unit 350 may store the light absorption coefficients of the components within the skin as shown in FIG. 2A and the light absorption intensities and fluorescence intensities of the components within the skin as shown in FIG. 2B. The arithmetic unit 350 may apply the stored information to the measured spectrum including the reflected light spectrum, the fluorescence spectrum, and the scattering light spectrum which are actually measured through the light detector 320 or the camera 330, and calculate the evaluation result including the oxidation degree and hydration degree of the skin or the kinds and concentrations of the components within the skin.

In another implementation, the arithmetic unit 350 may map the light information received by the light detector 320 to the images of the respective detection regions 30 of the skin. Through this operation, the arithmetic unit 350 may calculate the evaluation result for each detection region 30, which includes the oxidation degree and hydration degree of the skin or the kinds and concentrations of the components within the skin.

The control device 340 may control the above-described operations of the light source 310, the light detector 320, and the camera 330. For example, the control device 340 may control the arrangement of the light source 310, the light detector 320, and the camera 330, determine whether the scan function is normally performed, or adjust the operation sequence or timing of the light source 310, the light detector 320, and the camera 330. Furthermore, the control device 340 may control the arithmetic unit 350 to perform calculating using the light information acquired from the light detector 320 or the camera 330. Furthermore, the control device 340 may control a display device to display the calculation result.

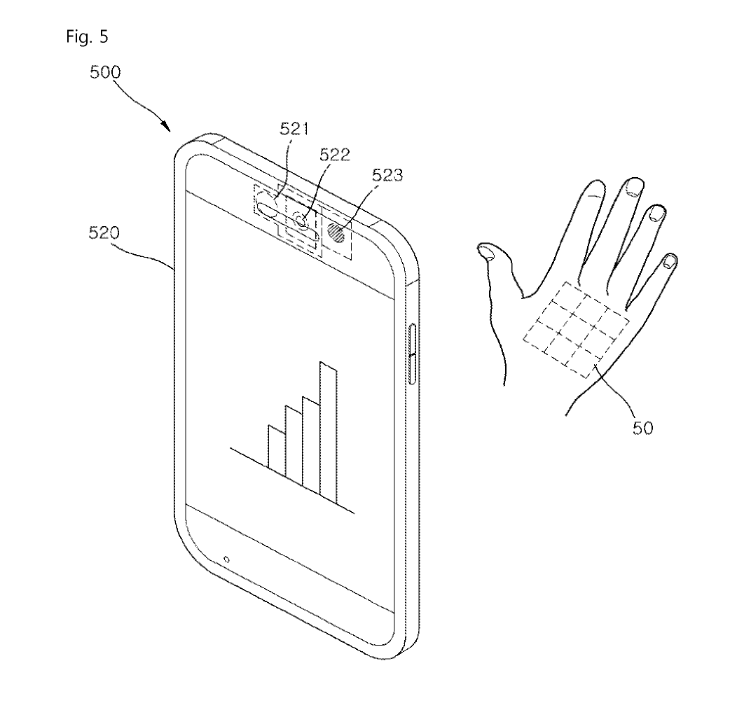

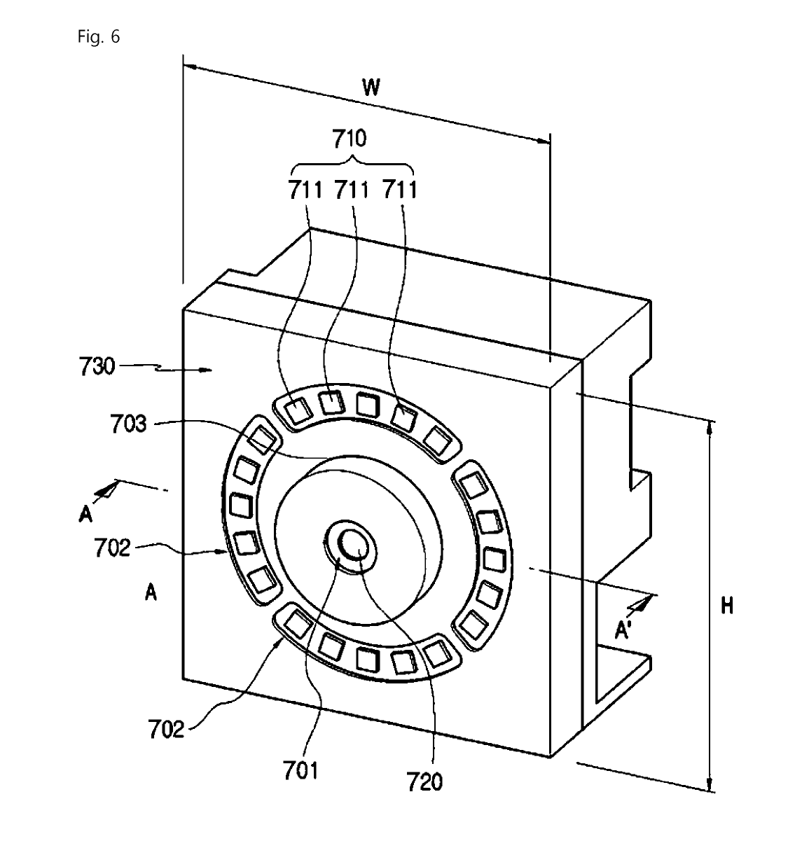

FIG. 5 is a schematic view of the skin condition evaluation apparatus according to another implementation of the disclosed technology. Referring to FIG. 5, the skin condition evaluation apparatus 500 may include a device body 520 having a light source 521, a light detector 522, and a camera 523. The device body 520 may further include a central processing unit and a control device therein. The device body 520 may further include a communication unit. The communication unit may include a connection module capable of connecting to a wired/wireless network, and transmit information stored in the device body 520 to outside or receive information from outside. The device body 520 may include a common smart phone, for example.

Referring to FIG. 5, detection regions 50 of skin to be evaluated may be placed in front of the portable skin condition evaluation apparatus 500. The detection regions 50 may be substantially the same as the detection regions 30 described above with reference to FIG. 4.

The light source 521 may include an LED capable of emitting one or more of visible light, UV light, or infrared light. The light source 521 may be arranged to irradiate light onto the detection regions 50. In some implementations, the light source 521 may be exposed to one surface of the device body 520 facing the detection regions 50.

The light detector 522 may include an optical diode, and detect one or more of scattering light, reflected light, or fluorescence emitted from the detection regions 50.

The camera 523 may include an optical diode and an image sensor, and capture an image of the detection regions 50.

The control device may include an application program to control the operation of the light source 521 which irradiates light onto the detection regions 50. Furthermore, the application program may control the light receiving operations of the light detector 522 and the camera 523. The arithmetic unit may process information of the received light, and finally determine the evaluation results such as the existences and concentrations of the components within the skin.

The portable skin condition evaluation apparatus 500 may include a storage device for storing the evaluation result of the skin condition therein. Furthermore, the portable skin condition evaluation apparatus 500 may include a display device to display the evaluation result of the skin condition.

FIG. 6 is a perspective view of a skin condition evaluation apparatus including LEDs according to some implementations of the disclosed technology. FIG. 7 is a cross-sectional view taken along line A-A′ of FIG. 6.

The skin condition evaluation apparatus including LEDs according to some implementations of the disclosed technology may include a light source 710 and a light detector 720 with a camera. The light source 710 may include a plurality of LEDs 711 configured to emit different wavelengths of light. The plurality of LEDs 711 may sequentially or non-sequentially emit light. For example, the plurality of LEDs 711 may irradiate light while alternately being turned on for each time period. In some implementations, the wavelength of light irradiated by each of the LEDs 711 may be selected in a wavelength range of 200 nm to 1,500 nm. FIG. 6 illustrates one example in that the LEDs 711 are arranged along a circle having the light detector 720 positioned at the center of the LEDs 711. The number of LEDs 711, the arrangement of the LEDs 711, and the size and shape of the LEDs 711 may not be limited to those illustrated in FIG. 6.

The light detector 720 may receive light which is reflected from the skin after the plurality of LEDs 711 of the light source 710 irradiates light. For this operation, the light detector 720 may include a CCD (Charge Coupled Device) imaging device, a CMOS (Complementary Metal-Oxide Semiconductor) imaging device, or another suitable imaging device. In some implementations, the light detector 720 may be positioned at the center of the LEDs 711 such that different wavelengths of light emitted from the respective LEDs 711 and reflected from the skin can be received at the same intensity by the light detector 720.

The light detector 720 may include a camera integrated therein. Thus, the light detector may not only detect light emitted from the skin, but also capture an image of the skin.

Various wavelengths of light may be sequentially or non-sequentially irradiated onto the skin by the plurality of LEDs 711 and then reflected from the skin. The light detector 720 may sequentially or non-sequentially receive the respective wavelengths of the reflected light, and capture an image of the skin. In some implementations, the light detector 720 may be configured to capture an image of the skin in synchronization with each of the LEDs 711, while light is irradiated by the LED 711. As a result, a plurality of images corresponding to the respective LEDs 711 having the respective wavelengths may be obtained through the light detector 720. In another implementation, while the plurality of LEDs 711 are sequentially turned on, the light detector 720 may be configured to consecutively capture images of the skin until all of the LEDs 711 are turned on. In this case, the images which are consecutively captured by the light detector 720 may be post-processed to obtain the plurality of images for each wavelength.

The skin condition evaluation apparatus including LEDs according to some implementations of the disclosed technology may be used to capture an image of the skin of an object. In a human or animal body, light reflected from the skin of the human or animal body may include various pieces of information related to the health condition of the human or animal body. Various factors including the color of the skin, the elasticity of the skin, and the number and size of blemishes or wrinkles existing in the skin may be set as observation targets for beauty treatment. When the skin condition evaluation apparatus including LEDs according to some implementations of the disclosed technology is used, skin images for each wavelength can be obtained. Thus, distribution of various components within the skin or oxygen distribution within blood vessels adjacent to the skin can be measured.

For example, various components existing in the skin including water, melanin, lipid, collagen, elastin, oxyhemoglobin or deoxyhemoglobin are known to have an influence on an absorption rate of light irradiated onto the skin with a specific wavelength. The distribution of collagen and melanin within the skin, the distribution of oxyhemoglobin and deoxyhemoglobin within the dermis, the thickness of the epidermis of the skin, and the water content of the skin can be used to indicate skin elasticity, skin darkness, oxygenation, age and/or sex, and dehydration, and have an influence on a spectrum of the reflected light in a specific wavelength range.

Thus, the skin condition evaluation apparatus including LEDs may determine the wavelengths of lights irradiated from the plurality of LEDs 711, based on the kinds of components within the blood vessels or skin to be evaluated. Using collagen, melanin, oxyhemoglobin, deoxyhemoglobin, the epidermis thickness, and the water content as indicators, the skin condition evaluation apparatus may irradiate lights having wavelengths corresponding to the indicators onto the skin, and measure the reflectance or diffuse reflectance of light reflected from the skin, thereby determining the distribution of the corresponding components within the skin or blood vessels.

At this time, the skin condition evaluation apparatus may irradiate UV light to firstly analyze specific component existing in the epidermis based on Equations 1 to 3, and remove the influence of the specific component in the epidermis during the additional light irradiation and analysis steps. Thus, it is possible to more exactly determine other components.

In some implementations, the skin condition evaluation apparatus may further include an LED 711 configured to irradiate light for data correction or other purposes such as the above-described melanin analysis, which is one of specific component in the epidermis.

In some implementations, each of the light detector 720 and the light source 710 may further include a polarizer (not illustrated) configured to adjust the polarization direction of light. The polarizer may be implemented in various forms. For example, the polarizer may include a polarizing plate, a polarizing filter, and a polarizing film. The polarizer may polarize light irradiated from the light source 710 and reflected light received by the light detector 720 in a specific direction. The polarizer may be detachably coupled to the light detector 720 or the light source 710. Furthermore, a user may rotate the polarizer coupled to the light detector 720 and/or the light source 710 so as to adjust the polarization direction.

In some implementations, the skin condition evaluation apparatus including LEDs may further include a body 730 to which the light detector 720 and the light source 710 are coupled. The body 730 may have a first opening 701 through which the light detector 720 receives light from outside. Furthermore, the body 730 may further have a second opening 702 through which light is irradiated to the outside from the light source 710. The body 730 may include a plurality of second openings 702 based on the number of LEDs 711, or the plurality of LEDs 711 may irradiate light through one second opening 702.

In some implementations, an optically transparent material such as glass may be coupled to each of the openings 701 and 702.

In some implementations, the body 730 may further include a cover 703 positioned to cover the light detector 720 and regions adjacent to the light detector 720. At this time, the first opening 701 may be formed in the cover 703 so as to be aligned with the light detector 720.

In some implementations, the light source 710 may include a substrate 713 to support the plurality of LEDs 711, and the LEDs 711 may be positioned over the substrate 713. For example, the substrate 713 may include a PCB (Printed Circuit Board), but is not limited thereto. The substrate 713 may be positioned in the body 730 and coupled to the body 730 such that the LEDs 711 over the substrate 713 are aligned with the second openings 702. The light detector 720 and the light source 710 may be coupled to the body 730 through various coupling members, and the detailed descriptions thereof are omitted.

Although not illustrated in the drawings, the skin condition evaluation apparatus including LEDs according to some implementations of the disclosed technology may further include a control circuit, a power supply unit and the like. The control circuit and the power supply unit may be positioned within the body 730 in a state where they are electrically connected to the light detector 720 and the light source 710. The power supply unit may be implemented in the form of a battery, or configured to receive power from an external power source through a wired connection.

The skin condition evaluation apparatus including LEDs according to some implementations of the disclosed technology may have a size to be easily carried by a user. For example, the body 730 may have a width W of about 110 mm and a height H of about 120 mm. Furthermore, the body 730 may have a thickness T of about 58 mm. Furthermore, the cover 703 covering the light detector 720 may have a thickness t of about 10 mm, and the light detector 720 may have a diameter r of about 54.5 mm. However, the above-described values are only examples. The specific shapes and values of the respective parts of the skin condition evaluation apparatus including LEDs may be set differently from those described in this specification, according to the sizes and shapes of the components forming the skin condition evaluation apparatus.

The skin condition evaluation apparatus may further include a driving device and a control device, in addition to the light detector 720 and the light source 710. In some implementations, the light detector 720 may include a first polarizer, and the light source 710 may include a second polarizer. The first and second polarizers may be configured to polarize light passing through the first and second polarizers in a specific direction. The polarization direction of the first polarizer may be referred to as a first direction, and the polarization direction of the second polarizer may be referred to as a second direction. The polarization of light reflected from the skin is affected by the position at which the light is reflected. When light is reflected from the skin surface, the polarization direction thereof is not changed or relatively less changed. When light is reflected after penetrating the skin to a predetermined depth, the polarization direction thereof is changed in comparison to when the light is incident. Thus, the polarization directions of the first and second polarizers may be properly adjusted to obtain desired information of the skin using the reflected light.

In some implementations, the first and second directions may be set to be parallel to each other. At this time, the light irradiated from the LEDs 711 may be polarized in a specific direction while passing through the second polarizer, and then irradiated onto an object. Furthermore, as the light reflected from the object passes through the first polarizer, only a component of the reflected light, corresponding to the specific direction, may be received by the light detector 720. In this case, the light detector can easily detect light which is reflected from the skin surface such that the polarization direction is not changed or relatively less changed while the light is reflected from the skin.

In another implementation, the first and second directions may be set to be not parallel from each other. For example, the first and second directions may be set to cross each other at right angles. At this time, the light irradiated by the LED 711 may be polarized in a specific direction while passing through the second polarizer, and then irradiated onto an object. However, when reflected light is detected, the reflected light may be passed through the first polarizer. Then, only a component corresponding to a polarization direction different from the specific direction may be received by the light detector 720. In this case, the light detector 720 can detect light which penetrates into the skin to a predetermined depth and is then reflected within the skin such that the polarization direction thereof is rotated by 90 degrees or changed in a different manner.

In another implementation, light including polarized components in all directions may be irradiated onto the object, without using the first and second polarizers, and reflected light including polarized components in all directions may be measured.

A driving unit may be electrically connected to the light source 710 and the control device. The driving unit may be operated according to the control of the control device, and provide a driving signal for turning on the plurality of LEDs 711. In some implementations, the driving unit may provide independent driving signals to the respective LEDs 711. The plurality of LEDs 711 may be configured to irradiate different wavelengths of light, and a light intensity, wavelength change, or current suitable for skin imaging and condition measurement may differ depending on each wavelength. The driving unit may separately transmit driving signals having electrical characteristics optimized for the respective LEDs 711 such that optimal measurement results can be obtained during an imaging process using a plurality of wavelengths.

The control device may be electrically connected to the driving unit and the light detector 720, and include a microprocessor or another suitable processing unit. For example, the control device may include a single-board computer, but is not limited thereto. The control device may control the driving unit to transmit a driving signal of which timings are controlled to sequentially or non-sequentially turn on the plurality of LEDs 711. Furthermore, the control device may control the light detector 720 to receive lights which are irradiated from the plurality of LEDs 711 and then reflected from the skin. For example, the control device may synchronize the imaging time of the light detector 720 with each of the driving signals. In some implementations, the control device may control the arithmetic unit to analyze light information or an image taken by the light detector 720, thereby obtaining information of the object for each wavelength.

In some implementations, the skin condition evaluation apparatus including LEDs may further include a display device. The display device may display an image of the object, obtained through the light detector 720, light information corresponding to each region of the image, and a skin component calculated using the light information. Furthermore, the display device may display a graphic user interface which enables a user to control the operation of the skin condition evaluation apparatus including LEDs. The display device may include a display unit such as an LCD (Liquid Crystal Display), but is not limited thereto.

According to some implementations of the disclosed technology, the skin condition evaluation apparatus and method may irradiate light onto skin, and receive light emitted from the skin, thereby easily evaluating the skin condition in a non-destructive manner. For example, UV light may be used as the light. Since the UV light is easily distinguished from visible light in the natural environment, the UV light can increase the reliability of the light signal received from the skin. Furthermore, the UV light does not deeply penetrate into the skin but disappears while being scattered, reflected, or absorbed in the epidermis or true skin. Thus, when the UV light is analyzed, the distribution of specific component, such as melanin, in the epidermis can be exactly analyzed. The light can be received through at least one of the light detector or the camera, and the light information for the respective detection regions of the skin can be obtained. Thus, the skin condition can be subdivided and evaluated with more accuracy. Furthermore, as the plurality of LEDs sequentially or non-sequentially irradiate different wavelengths of light, the information of skin for each wavelength can be obtained. Furthermore, the polarizer may be used to reduce distorted information, and the polarization directions of irradiated light and reflected light may be set to parallel polarization, cross polarization, or non-polarization modes. Thus, the evaluation can be accurately performed. Furthermore, as the plurality of LEDs are operated according to separate driving signals, the electrical characteristics of the respective LEDs for each wavelength can be controlled to obtain the same light intensity.

While various embodiments have been described above, it will be understood to those skilled in the art that the embodiments described are by way of example only. Accordingly, the disclosure described herein should not be limited based on the described embodiments.

What is claimed is:

1. A skin condition evaluation method comprising:
providing a light source including one or more light emitting diodes (LEDs);
irradiating light from the light source onto a skin;
responsive to the irradiating, receiving, by a light detector, light emitted through the skin, the received light emitted through the skin including ultraviolet (UV) light; and
calculating, based at least partly on the received light emitting through the skin, an amount of a component distributed in the skin.

2. The skin condition evaluation method of claim 1, wherein, based on a presence of the component in an epidermis of the skin, the calculating of the amount of the specific component comprises:
calculating a diffuse reflectance R of a semi-infinite layer based at least partly on information on light received by the light detector;
calculating a single scattering albedo ω(λ) from the diffuse reflectance R of the semi-infinite layer by
$$R = ( 1 - {ρ}_{01} ) [ 1 - {\hat{ρ}}_{10} ( ω ) ] \frac{{\hat{R}}_{d} ( ω )}{1 - {\hat{ρ}}_{10} ( ω ) {\hat{R}}_{d} ( ω )} ,$$
where ρ₀₁ is the specular reflection of incident radiation by the surrounding/medium interface, \hat{ρ}₁₀ is semi-empirical hemispherical-hemispherical reflectivity, and \hat{R}_{d} is the semi-empirical diffuse reflectance of semi-infinite layer when exposed to diffuse irradiation;
calculating an absorption coefficient μₑₚᵢ of the epidermis from the single scattering albedo ω(λ) using an equation,
$$ω ( λ ) = \frac{{μ}_{s , tr} ( λ )}{{μ}_{s , tr} ( λ ) + {μ}_{epi}} ,$$
where μ_{s, tr} is a transport scattering coefficient; and
calculating a volume fraction fₛₚₑ of specific component in the epidermis from the absorption coefficient μₑₚᵢ of the epidermis using an equation,
μₑₚᵢ=μₛₚₑ(λ)fₛₚₑ+μ_{back}(λ)(1−fₛₚₑ), where μback is background absorption of human flesh.

3. The skin condition evaluation method of claim 1, wherein the UV light has a peak wavelength of 300 nm to 400 nm.

4. The skin condition evaluation method of claim 1, wherein the irradiating of the light includes emitting by the one or more LEDs one or more lights including UV light, visible light, or infrared light, and
the skin condition evaluation method further comprises removing a contribution of the amount of the component to the light received by the light detector.

5. The skin condition evaluation method of claim 1, wherein the irradiating of the light onto the skin comprises providing different wavelengths of light emitted from a plurality of LEDs, and
the skin condition evaluation method further comprises removing a contribution of the amount of the specific component to the light received by the light detector.

6. The skin condition evaluation method of claim 1, wherein the receiving of the light emitted through the skin comprises receiving one or more of a reflected light spectrum, a fluorescence spectrum, or a scattering light spectrum from the skin.

7. The skin condition evaluation method of claim 1, further comprising calculating a spectrum absorbed by the component of the skin based on the received light, using an arithmetic unit.

8. The skin condition evaluation method of claim 1, wherein the irradiating of the light onto the skin comprises emitting by the one or more LEDs a polarizing light.

9. The skin condition evaluation method of claim 8, wherein the receiving of the light emitted through the skin comprises receiving polarized light of the light emitted from the skin.

10. The skin condition evaluation method of claim 1, further comprising:
capturing an image of the skin, the captured image of the skin being divided into regions;
mapping information associated with the received light from the light detector to the respective regions; and
displaying the information mapped to the respective regions of the skin image, using a display device.

11. The skin condition evaluation method of claim 1, wherein the component is melanin.

12. A skin condition evaluation apparatus comprising:
a light source comprising one or more LEDs to irradiate light including UV light onto a skin;
a light detector configured to receive light emitted through the skin responsive to the irradiated light; and
an arithmetic unit configured to calculate a spectrum absorbed by one or more components of the skin based at least partly on the received light,
wherein each of the one or more LEDs is independently driven to irradiate the light, and
wherein the arithmetic unit is configured to calculate an amount of the one or more components of the skin based on the light received by the light detector, and remove a contribution of the calculated amount of the one or more components to the received light.

13. The skin condition evaluation apparatus of claim 12, wherein the light detector receives one or more of a reflected light spectrum, a fluorescence spectrum, or a scattering light spectrum from the skin.

14. The skin condition evaluation apparatus of claim 12, wherein the light source comprises a polarizing device to polarize the light emitted from the one or more LEDs such that the polarized light is irradiated on the skin.

15. The skin condition evaluation apparatus of claim 14, wherein the light detector comprises a polarizing device to receive the polarized light of the light emitted from the skin.

16. The skin condition evaluation apparatus of claim 15, wherein an angle between a polarization direction of the polarizing device of the light source and a polarization direction of the polarizing device of the light detector is adjustable.

17. The skin condition evaluation apparatus of claim 12, further comprising a camera configured to capture an image of the skin,
wherein the arithmetic unit maps the light information received by the light detector to a plurality of regions of the skin image captured through the camera, and
the skin condition evaluation apparatus further comprises a display device configured to display the light information mapped to the respective regions of the skin image.

18. The skin condition evaluation apparatus of claim 17, wherein the light detector and the camera are integrated with each other.

19. The skin condition evaluation apparatus of claim 12, wherein the one or more LEDs are symmetrically or radially arranged with respect to the light detector.

20. The skin condition evaluation apparatus of claim 12, wherein the one or more components include melanin.

21. A skin condition evaluation method comprising:
receiving, by a light detector, light emitted through a person's skin under optical illumination, the received light including ultraviolet (UV) light;
processing a detector output signal from the light detector to determine an amount of a component distributed in an epidermis of the skin based at least partly on information of the skin that is contained in the received light; and
using the determined amount of the component distributed in the epidermis of the skin to evaluate a skin condition.

22. The method as in claim 21, wherein the amount of the component distributed in the epidermis of the skin includes an oxidation degree, a hydration degree, or a collagen level.
